(19) United States
(12) Patent Application Publication
(10) Pub. No.: US 2025/0380933 A1
(43) Pub. Date: Dec. 18, 2025

(54) SURGICAL SYSTEM

(71) Applicant: PELETON SURGICAL, LLC, SCOTTSDALE, AZ (US)

(72) Inventor: Andrew J. Bala, Scottsdale, AZ (US)

(21) Appl. No.: 19/241,891

(22) Filed: Jun. 18, 2025

Related U.S. Application Data
(60) Provisional application No. 63/661,395, filed on Jun. 18, 2024.

(51) Int. Cl. A61B 17/00; A61B 50/30

(52) U.S. Cl. CPC A61B 17/00; A61B 50/30; A61B 2017/00199; A61B 2017/00225; A61B 2017/0023; A61B 2017/00477

(57) ABSTRACT

A surgical system includes a console. A tool assembly is operable to be coupled with the console. The tool assembly can be operable to be utilized for a surgical procedure. The tool assembly can be configured to be single-use.

### CROSS REFERENCE TO RELATED APPLICATIONS

This application claims the benefit of U.S. Provisional Patent Application Ser. No. 63/661,395, filed on Jun. 18, 2024, and titled “SURGICAL SYSTEM,” of which is incorporated by reference herein in its entirety.

### FIELD

The present disclosure relates generally to surgical systems with single-use tool assemblies.

### BACKGROUND

The rapid increase in the number of Ambulatory Surgical Centers across the US, and the growing number of doctors planning to perform minor surgical procedures in their own office space, has highlighted several challenging areas that are preventing even faster growth of these cost-effective changes to the way healthcare can be delivered for many more patients, at a more economical cost.

### BRIEF DESCRIPTION OF THE DRAWINGS

Implementations of the present technology will now be described, by way of example only, with reference to the attached figures, wherein:

FIG. 1 illustrates a perspective view of a surgical system in a surgical environment.

FIG. 2 illustrates a console for the surgical system.

FIG. 3 illustrates a control component for the surgical system.

FIGS. 4A, 4B, and 4C illustrate a tool assembly such as a hand drill assembly.

FIGS. 5A, 5B, and 5C illustrate a tool assembly such as a handpiece assembly.

FIGS. 6A, 6B, and 6C illustrate a tool assembly such as a percutaneous assembly.

FIGS. 7A, 7B, and 7C illustrate a tool assembly such as a saw blade assembly.

FIG. 8 is a schematic diagram of a controller which may be employed as shown in FIGS. 1-7C.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

Several definitions that apply throughout this disclosure will now be presented. The term “coupled” is defined as connected, whether directly or indirectly through intervening components, and is not necessarily limited to physical connections. The term “substantially” is defined to be essentially conforming to the particular dimension, shape or other word that substantially modifies, such that the component need not be exact. For example, substantially cylindrical means that the object resembles a cylinder, but can have one or more deviations from a true cylinder. The term “about” means reasonably close to the particular value. For example, about does not require the exact measurement specified and can be reasonably close. As used herein, the word “about” can include the exact number. The term “near” as used herein is within a short distance from the particular mentioned object. The term “near” can include abutting as well as relatively small distance beyond abutting. The terms “comprising,” “including” and “having” are used interchangeably in this disclosure. The terms “comprising,” “including” and “having” mean to include, but not necessarily be limited to the things so described.

Most surgery centers and doctors' offices do not have the capability to reprocess and/or sterilize larger power tool trays. Capital equipment costs from legacy suppliers are of course a huge barrier for small clinics and doctors with limited resources. As are the costs of having inventory of sufficient back up accessories in stock to ensure all necessary equipment is readily available 24/7 to handle any emergency.

The inconvenience of having to source reliable and efficient contractors to provide cleaning and sterilisation services for the (post operation) used equipment. This is a critical and costly process that is necessary to prevent cross contamination of patients and avoid the massive financial penalties that can be incurred if patients are injured as the result of such cross contamination.

Missing or broken components after the equipment is returned from the cleaning contractor, often results in costly time delays, or even cancellation of operations if replacements are not readily available.

The cost-effective surgical system disclosed herein provides everything the operator will need to perform surgery in the Ambulatory Surgery Centers, Clinics, or dedicated aseptic space within their office. In some examples, the surgical system can be utilized for small bone procedures. In some examples, the surgical system can provide one or more high-speed tool assemblies that can be single-use. In some examples, the tool assembly can be disposable.

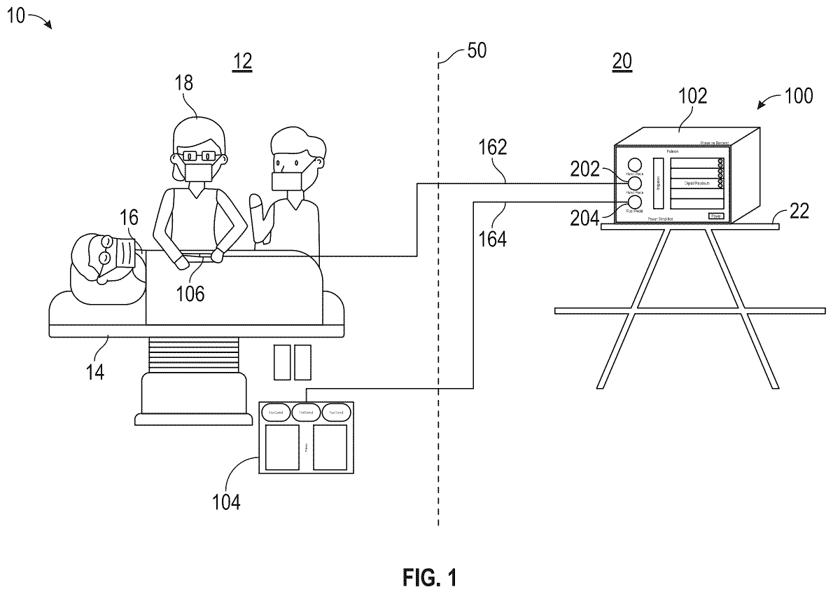

The disclosure now turns to FIG. 1, which illustrates an example of a surgical system 100. As shown in FIG. 1, the system 100 can be utilized in a surgical environment 10 (e.g., an operating room). The surgical environment 10 includes a sterile field 12 and a non-sterile field 20. The sterile field 12 and the non-sterile field 20 can be separated by a dividing line 50. In at least one examples, the dividing line 50 can include a physical barrier such as a sterile drape or surgical draping, to delineate the sterile field 12 from the non-sterile field 20. The sterile field 12 includes the operating table 14, the patient 16, and the operator 18 operating on the patient 16. The sterile field 12 must remain sterile to prevent contamination, which could introduce harmful pathogens and compromise the safety of surgical procedures or medical treatments. The non-sterile field 20 does not have to be sterile and can include a surface 22 (e.g., a table). For example, the surface 22 can be operable to receive tools and/or packaging that is not sterile.

The system 100 can include a console 102 and one or more tool assemblies 106. Each of the one or more tool assemblies 106 can be communicatively coupled with the console 102. In some examples, the tool assembly 106 can be detachably coupled with the console, for example via a tool conduit 162. In some examples, the tool assembly 106 can be wirelessly coupled with the console 102 such that signals and/or instructions can be communicated wirelessly between the console 102 and the tool assembly 106. In at least one example, the system 100 can include a control component 104 (e.g., a foot pedal) operable to be coupled with the console 102. In some examples, the control component can be operable to convert mechanical force exerted by an operator's foot into an electrical signal that controls the tool assembly 106 during operation. Accordingly, the signal from the control component 104 can be passed to the console 102 which then provides a signal to the tool assembly 106 to cause the tool assembly 106 to perform one or more actions during the operation. In at least one example, the control component 104 can be detachably coupled with the console 102, for example via a control conduit 164 that is connected with the control connector 204 of the console 102. In some examples, the control component 104 can be wirelessly connected with the console 102 so that any input from the control component 104 can be communicated to the console 102.

Any or all components can be single use, sterile packed and ready to use, right out of the pack. Each sterile item is 100% sterile, fully functioning. In some examples, as illustrated in FIG. 1, the console 102 may not be sterile as the console 102 does not have to be in the sterile field 12 and can remain in the non-sterile field 20. In some examples, the control component 104 may not be sterile. For example, in some examples, the control component 104 may be provided on the floor and controlled by the operator's foot. In some examples, the console 102 and/or the control component 104 can be fully reusable to reduce costs and can remain in the operative room for future use.

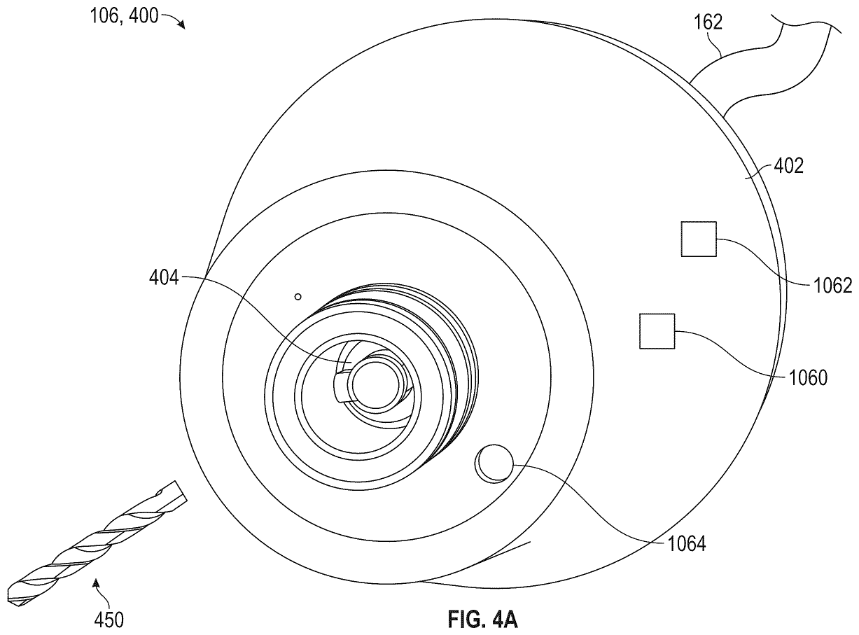

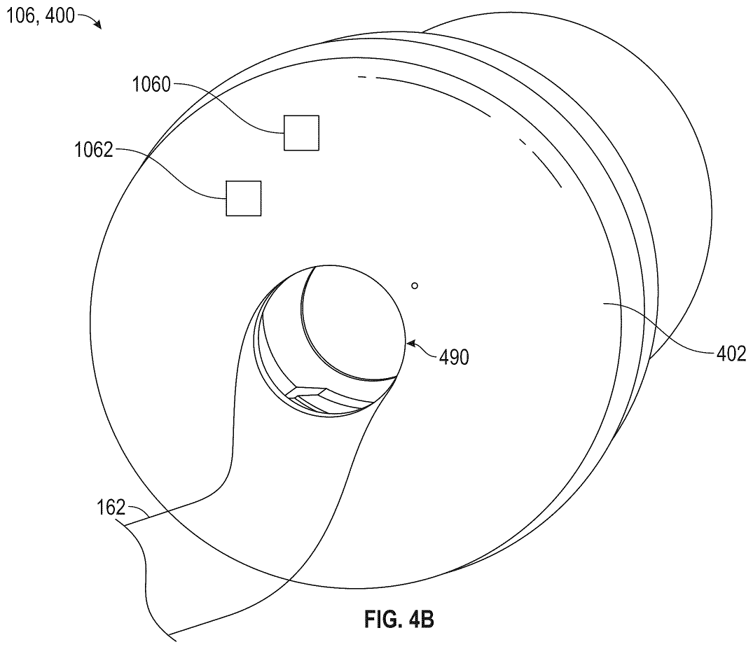

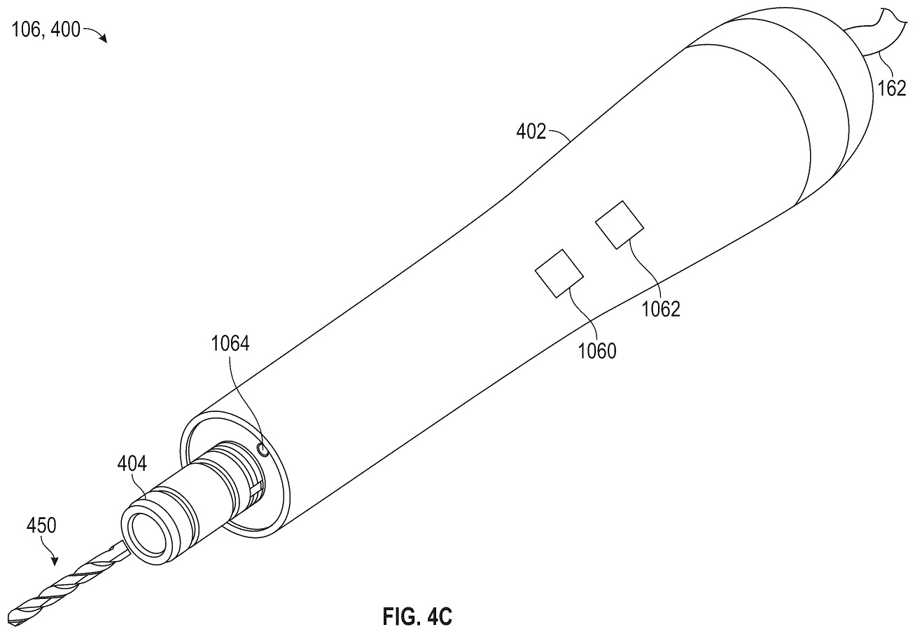

Meanwhile, the one or more tool assemblies 106 are each single-use, sterile packed and ready to use, right out of the pack. With such a configuration, the console 102 can remain in the surgical environment 10 in the non-sterile field 20 while any new tool assemblies 106 that need to be sterilized (for example for a new operation or another one is needed due to malfunction, etc.) can be easily replaced by connecting with the console 102 and immediately being able to be utilized during the surgery. The used tool assemblies 106 and/or control component 104 are then disposable. In some examples, the attachments 406, 506, 606, 706 (as shown in FIGS. 4A-7C) for the tool assembly 106 can also be sterile-packed, single-use, and disposable. In some examples, each of the console 102, the control component 104, and the tool assemblies 106 come sterile packed and ready to use for first-use.

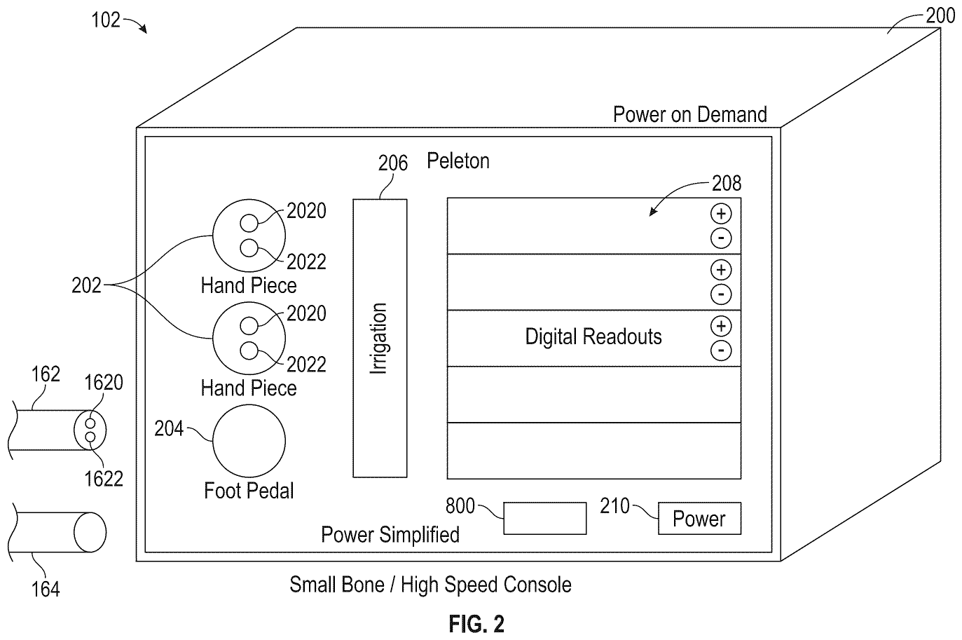

FIG. 2 illustrates the console 102. The console 102 can include a housing 200 that is operable to contain the components of the console 102. The console 102 can include connection(s) 202, 204 (e.g., for the tool conduit 162 and/or the control conduit 164) for the one or more tool assemblies 106 (for example marked in FIG. 2 as “Hand Piece”) and/or the control component 104 (for example marked in FIG. 2 as “Foot Pedal”). In at least one example, the console 102 can include a display 208 (for example marked in FIG. 2 as “Digital Readouts”) which can display readouts related to the tool assemblies 106, the foot pedal 104, and/or any other suitable sensors and/or equipment that can provide feedback to the console 102.

In at least one example, the console 102 can transmit signals between the tool assembly 106 and the console 102. For example, the console 102 can relay operating signals to the tool assembly 106 (e.g., from the control component 104) via the tool conduit 162. In some examples, the tool conduit 162 can include an electrical conduit 1620 that can be coupled with a corresponding electrical connection 2020 of a tool connector 202 of the console 102. The tool conduit 162 (e.g., via the electrical conduit 1620) can be operable to transmit signals between the console 102 and the tool assembly 106. The console 102 can control (e.g., via the control component 104) operations of each tool assembly 106 such as the on/off, right/left direction, irrigation and variable speed functions. In some examples, the console 102 can be operable to receive signals from the tool assembly 106, for example from sensors included in and/or coupled with the tool assembly 106. In some examples, the console 102 can determine that the tool assembly 106 needs adjustment based on feedback from the tool assembly 106, and the console 102 can send one or more signals to the tool assembly 106 to adjust as needed. The console 102 can include a controller 800 that can be operable to receive signals and/or data, make determinations based on the signals and/or data, and/or transmit signals and/or data.

In at least one example, the console 102 can provide irrigation to the one or more tool assemblies 106. For example, the console 102 can include an irrigation system 206 such as a fluid tank, pump, etc. The fluid can be transported to the tool assembly 106 via the tool conduit 162. For example, the tool conduit 162 can include an irrigation conduit 1622 that is operable to couple with an irrigation connector 2022 of the console 102. The irrigation conduit 1622 can be coupled via the irrigation connector 2022 to the irrigation system 206 of the console 102 and transport the fluid. Irrigation for the one or more tool assemblies 106 can help to clear away debris, blood, and tissue fragments from the surgical site, ensuring better visibility and reducing the risk of infection. The console 102 can be operable to retain the fluid needed for irrigation and provide the fluid to the tool assembly 106 during the operating procedure via the tool conduit.

In at least one example, the console 102 can transmit power to the one or more tool assemblies 106, for example via the tool conduit 162. In at least one example, the console 102 can include a power supply 210. In some examples, the console 102 can be powered via cable to a secure power source. In at least one example, the console 102 can include a battery as a power supply 210. The battery can be included that can be charged making it the easiest/most convenient to use power option. In some examples, the battery can be integrated into the console. In some examples, the battery can be removably coupled with the console 102 as a secondary power source. With the battery, the console 102 can function even if the permanent power source (e.g., building power) shuts down, and/or the console 102 can function and be utilized in remote locations without a permanent power source.

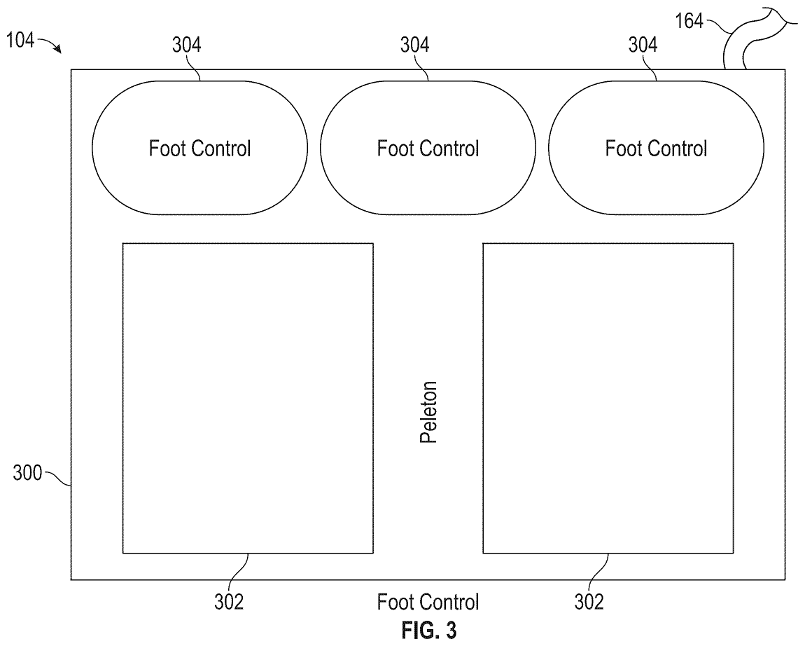

FIG. 3 illustrates a control component 104 (e.g., a foot pedal). The control component 104 can include one or more control mechanisms 304 that are operable to receive input from the operator to control settings for operation of the tool assembly 106. For example, the control mechanism 304 can change the direction of the tool assembly 106 movement. In some examples, the control mechanism 304 can control features such as light, irrigation, and/or air for the tool assembly 106. In at least one example, the control component 104 can include one or more activation mechanisms 302 to control the activation of the instructions for the tool assembly 106. For example, the activation mechanism 302 can receive input (e.g., stepped on and pushed down on) by the operator to control the tool assembly 106. In some examples, the activation mechanism 302 can provide an on or off signal. In some examples, the activation mechanism 302 can provide variable signals to control the strength of the action(s) performed by the tool assembly 106. The control component 104 can be operable to convert the mechanical force exerted by the operator's foot (for example by pressing onto the control mechanism 304 and/or activation mechanism 302) into an electrical signal that controls (e.g., activates, manipulates, adjusts, etc.) the one or more tool assemblies 106 during a procedure. The control component 104 can transmit the signal(s) to the console 102 via the control conduit 164, and the console 102 can then transmit the desired signal(s) to the relevant tool assembly 106 via the tool conduit 162.

FIGS. 4A-7C illustrate different examples of tool assemblies 106 that can be included in the system 100. While FIGS. 4A-7C illustrate some examples of tool assemblies 106, different tool assemblies 106 can also be utilized without deviating from the scope of the disclosure.

The one or more tool assemblies 106 can include ergonomically designed, variable speed hand pieces to which a wide range of attachments 450, 550, 650, 750 can easily be snapped into place. Accordingly, the attachments 450, 550, 650, 750 can be detachably coupled with each of the attachments 450, 550, 650, 750 to perform different actions during operation. All component parts are single use items, so there is no need for the reserialization process & costs related. Each of the tool assemblies 106 and corresponding attachments 450, 550, 650, 750 can be disposable.

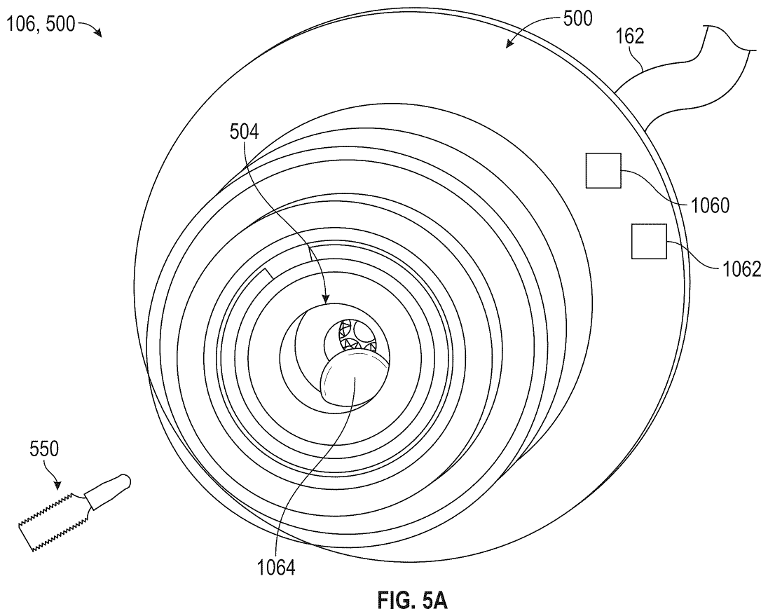

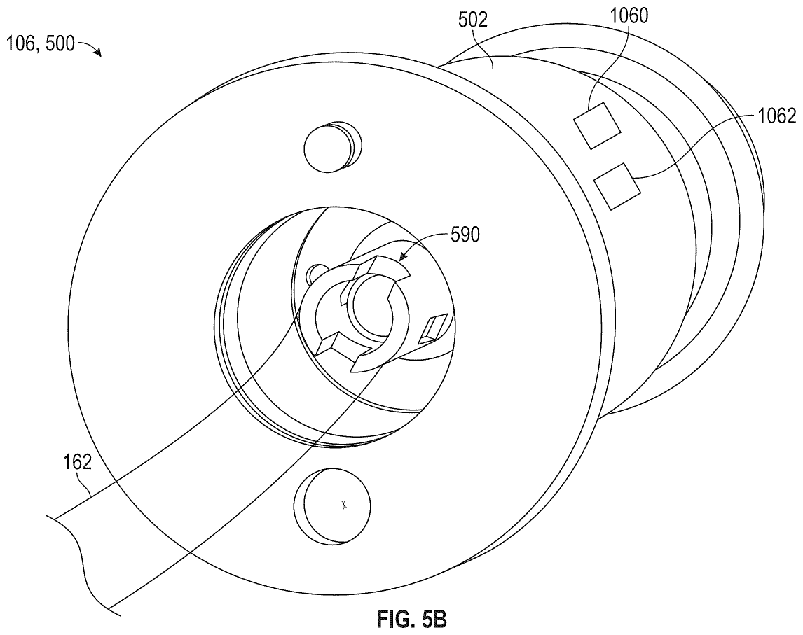

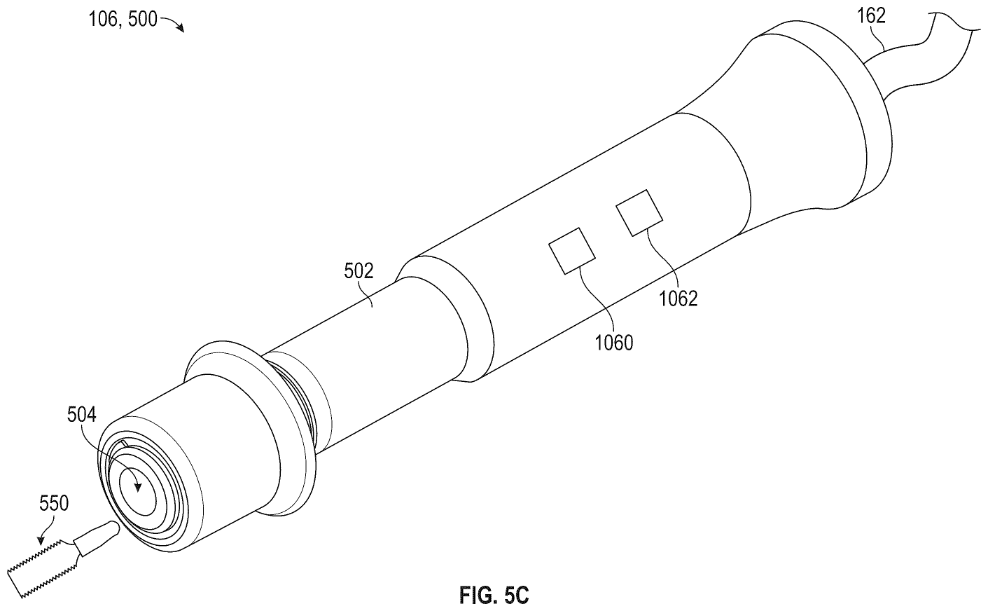

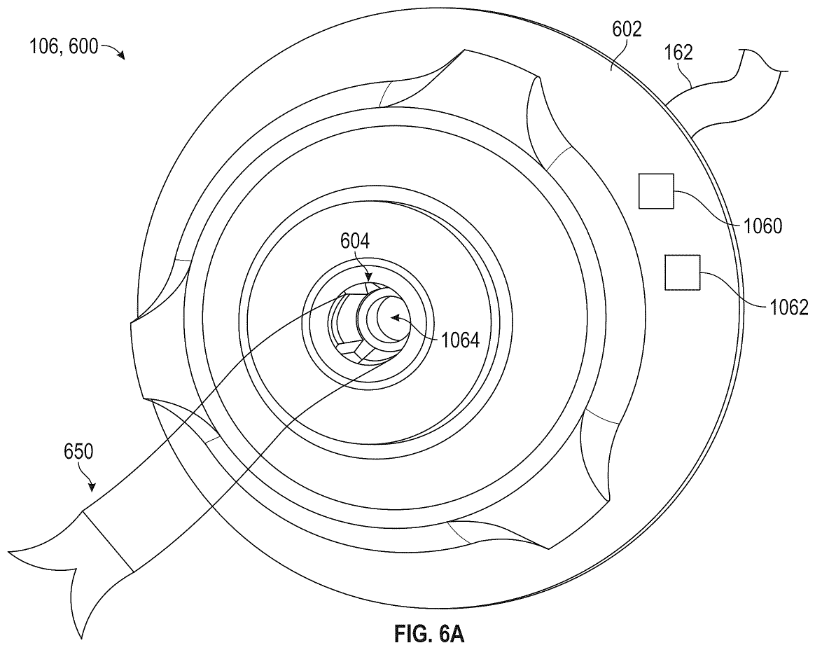

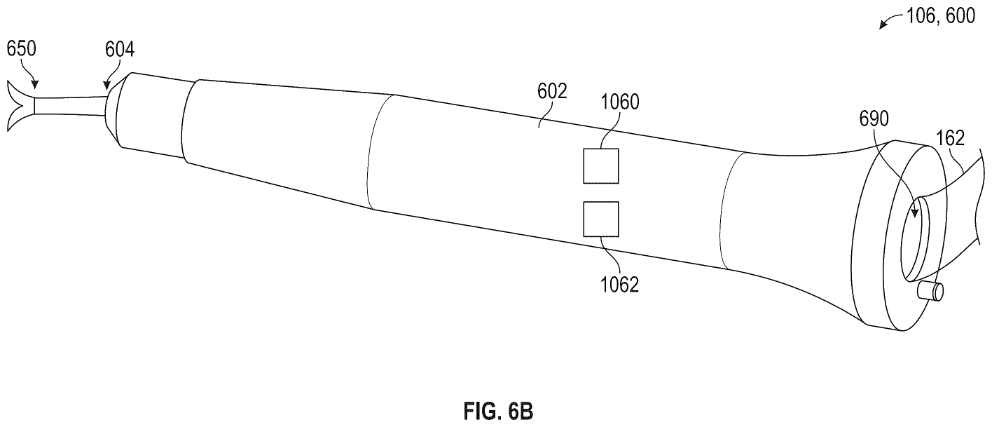

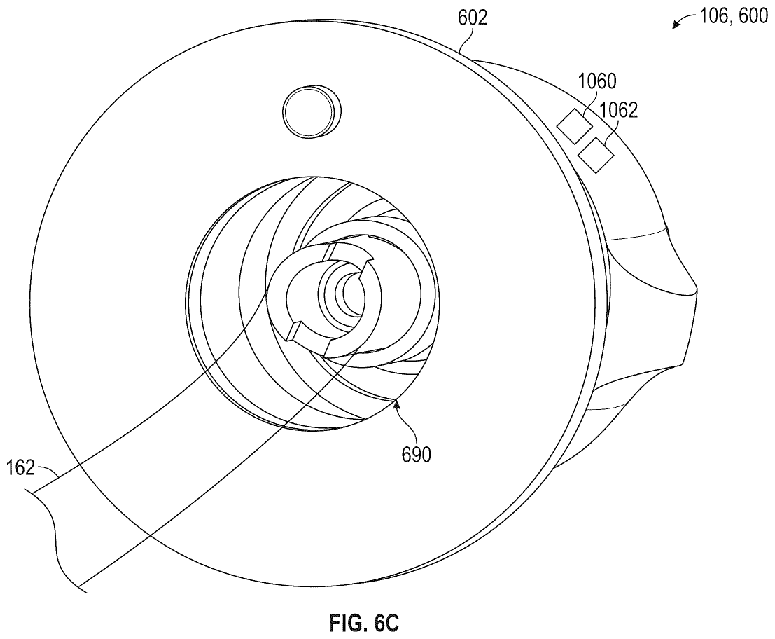

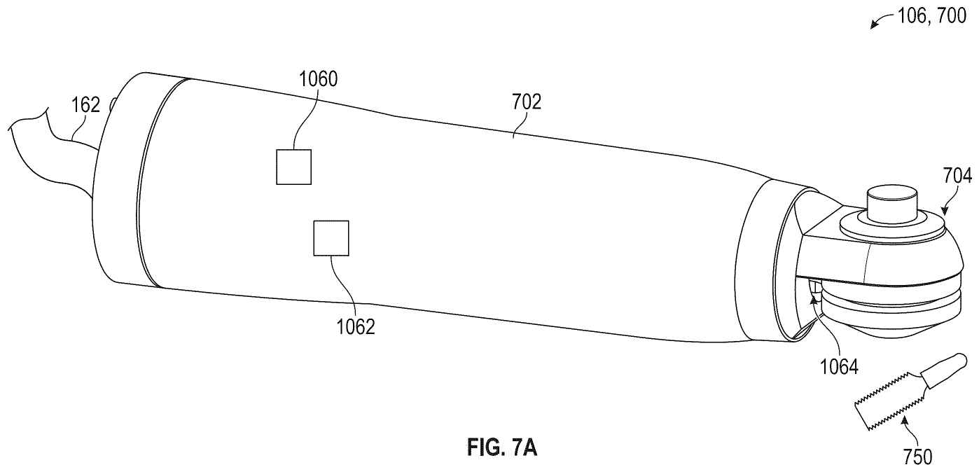

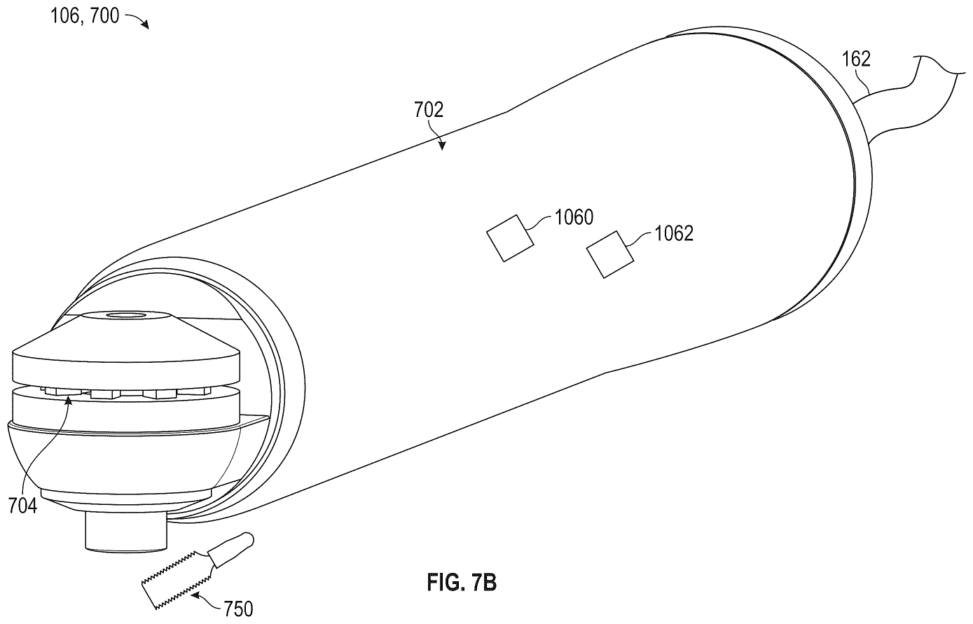

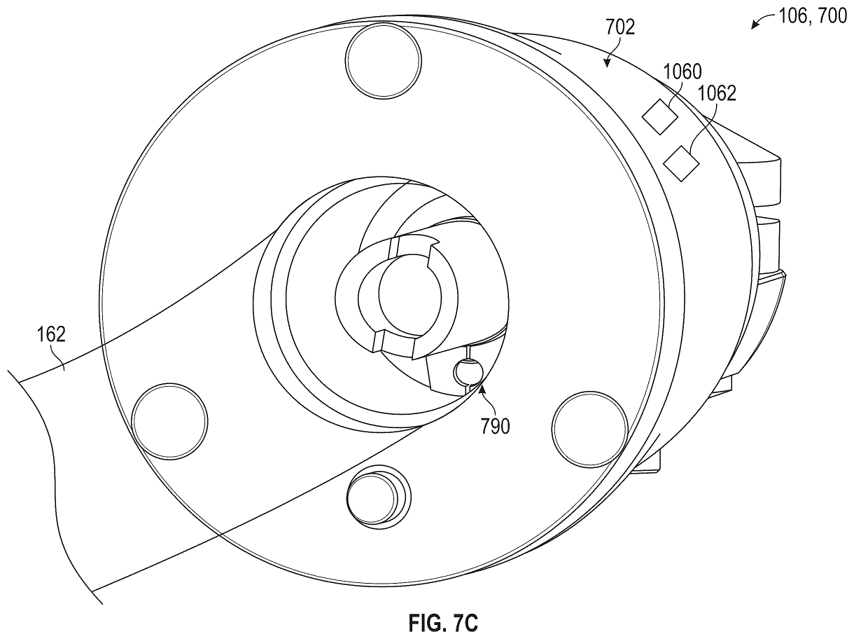

FIGS. 4A-4C illustrate a tool assembly 106, 400 that can be operable to receive an attachment 450, such as a drill bit, to precisely create holes in bone during the procedure. For example, the hand drill assembly 400 can include a body 402 that includes and/or is coupled with a receiving portion 404 that is operable to receive and couple with the attachment 450. As shown in FIG. 4C, the tool assembly 400 can include a connector 490 that is operable to be connected to the tool conduit 162. FIGS. 5A-5C illustrate a tool assembly 106 that can be operable to hold various attachments 550, for example surgical instruments such as drills, saws, or burrs, facilitating precise and controlled manipulation during procedures. For example, the tool assembly 500 can include a body 502 that includes and/or is coupled with a receiving portion 504 that is operable to receive and couple with the attachment 550. As shown in FIG. 5C, the tool assembly 500 can include a connector 590 that is operable to be connected to the tool conduit 162. FIGS. 6A-6C illustrate a tool assembly 600 (e.g., a percutaneous assembly) that can be operable to be used for minimally invasive procedures, enabling access to internal structures or organs through small incisions or puncture sites. For example, the tool assembly 600 can include a body 602 that includes and/or is coupled with a receiving portion 604 that is operable to receive and couple with the attachment 650 (e.g., a minimally invasive procedure tool). As shown in FIG. 6C, the tool assembly 600 can include a connector 690 that is operable to be connected to the tool conduit 162. FIGS. 7A-7C illustrate a tool assembly 700 (e.g., a saw blade assembly) that can be operable to receive attachments 750 such as saw blades to cut through bone during the procedures with precision and control. For example, the tool assembly 700 can include a body 702 that includes and/or is coupled with a receiving portion 704 that is operable to receive and couple with the attachment 750. As shown in FIG. 7C, the tool assembly 700 can include a connector 790 that is operable to be connected to the tool conduit 162.

While FIGS. 4A-7C illustrate different tool assemblies 106 for different functions and attachments 450, 550, 650, 750, in some examples, one tool assembly 106 can be operable to detachably couple with a plurality of different attachments 450, 550, 650, 750 to provide a wider range of actions that the single tool assembly 106 can be used for. For example, the attachments 450, 550, 650, 750 can be detachably coupled with the tool assembly 106, perform an action, be removed, and discarded. A different attachment 450, 550, 650, 750 can then be detachably coupled with the tool assembly 106 for the next action.

In at least one example, as shown in FIGS. 4A-7C, the tool assembly 106 can include an irrigation port 1064 that is operable to be in fluid communication with the irrigation 206 of the console 102. The irrigation port 1064 can permit the fluid to flow out of the tool assembly 106 to provide irrigation for the operation. In some examples, the irrigation port 1064 can be formed in the body 402, 502, 602, 702 of the tool assembly 106. In some examples, the irrigation port 1064 can be coupled with the tool assembly 106.

In some examples, as illustrated in FIGS. 4A-7C, the tool assemblies 106 can include one or more motors 1060 that can be operable to drive the attachments 450, 550, 650, 750 during operation. In at least one example, the motor(s) 1060 can receive power from the console 102 via the tool conduit 162. In some examples, the tool assembly 106 can include a battery 1062 to power the tool assembly 106 such that the tool assembly 106 does not need to be connected to a console 102 for power. The battery 1062 can then provide power to the motor(s) 1060 to drive the attachments 450, 550, 650, 750 or perform any desired action during operation.

Attachments 450, 550, 650, 750 can cover every action performed by the tool assembly 106 that expensive conventional, reusable equipment can offer, with the same, or in some instances, improved performance over generic systems. Some example actions covered include:
For any surgical procedure that requires drilling, reaming, and/or sculpting for bones, cartilage, screws, and/or implants.
Pin and wire placement for fixation.
Sawing for osteotomy of small bones.

The tool assemblies 106, attachments 450, 550, 650, 750, irrigation, tool conduit 162, and/or irrigation tubing can be packed individually in single use, sterile packaging. In some examples, the tool assemblies 106 can each be disposable after use. Accordingly, the operator can simply open the package and be ready to use the tool assembly 106 during operation in the sterile environment. The operator only has to connect the tool assembly 106 to the console 102, and the operation is ready to commence or continue. For example, after opening in the sterile field 12, the connecting ends of the tool conduit 162 can be passed off to a circulator/tech who can connect these ends to the console 102.

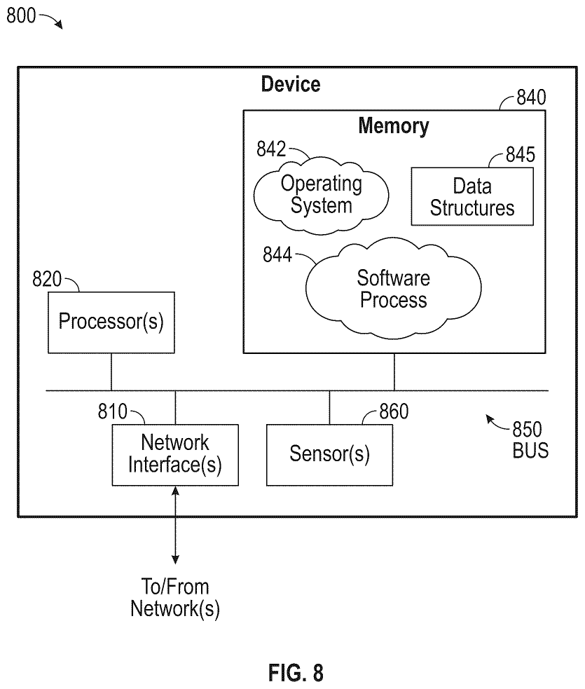

FIG. 8 is a block diagram of an exemplary controller 800. Controller 800 is configured to perform processing of data and communicate with other components, for example as illustrated in FIGS. 1-7C. In operation, controller 800 communicates with one or more of the components discussed herein and may also be configured to communication with remote devices/systems.

As shown, controller 800 includes hardware and software components such as network interfaces 810, at least one processor 820, sensors 860 and a memory 840 interconnected by a system bus 850. Network interface(s) 810 can include mechanical, electrical, and signaling circuitry for communicating data over communication links, which may include wired or wireless communication links. Network interfaces 810 are configured to transmit and/or receive data using a variety of different communication protocols, as will be understood by those skilled in the art.

Processor 820 represents a digital signal processor (e.g., a microprocessor, a microcontroller, or a fixed-logic processor, etc.) configured to execute instructions or logic to perform tasks in a surgical environment. Processor 820 may include a general purpose processor, special-purpose processor (where software instructions are incorporated into the processor), a state machine, application specific integrated circuit (ASIC), a programmable gate array (PGA) including a field PGA, an individual component, a distributed group of processors, and the like. Processor 820 typically operates in conjunction with shared or dedicated hardware, including but not limited to, hardware capable of executing software and hardware. For example, processor 820 may include elements or logic adapted to execute software programs and manipulate data structures 845, which may reside in memory 840.

Sensors 860 typically operate in conjunction with processor 820 to perform measurements, and can include special-purpose processors, detectors, transmitters, receivers, and the like. In this fashion, sensors 860 may include hardware/software for generating, transmitting, receiving, detection, logging, and/or sampling magnetic fields, seismic activity, and/or acoustic waves, or other parameters.

Memory 840 comprises a plurality of storage locations that are addressable by processor 820 for storing software programs and data structures 845 associated with the examples described herein. An operating system 842, portions of which may be typically resident in memory 840 and executed by processor 820, functionally organizes the device by, inter alia, invoking operations in support of software processes and/or services 844 executing on controller 800. These software processes and/or services 844 may perform processing of data and communication with controller 800, as described herein. Note that while process/service 844 is shown in centralized memory 840, some examples provide for these processes/services to be operated in a distributed computing network.

It will be apparent to those skilled in the art that other processor and memory types, including various computer-readable media, may be used to store and execute program instructions pertaining to the fluidic channel evaluation techniques described herein. Also, while the description illustrates various processes, it is expressly contemplated that various processes may be embodied as modules having portions of the process/service 844 encoded thereon. In this fashion, the program modules may be encoded in one or more tangible computer readable storage media for execution, such as with fixed logic or programmable logic (e.g., software/computer instructions executed by a processor, and any processor may be a programmable processor, programmable digital logic such as field programmable gate arrays or an ASIC that comprises fixed digital logic. In general, any process logic may be embodied in processor 820 or computer readable medium encoded with instructions for execution by processor 820 that, when executed by the processor, are operable to cause the processor to perform the functions described herein.

The embodiments shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the embodiments described above may be modified within the scope of the appended claims.

What is claimed is:

1. A surgical system comprising:
a console;
a tool assembly operable to be coupled with the console, the tool assembly operable to perform one or more actions for a surgical procedure,
wherein the tool assembly is configured to be sterile and single-use.

2. The surgical system of claim 1, wherein the tool assembly is detachably coupled with the console via a conduit.

3. The surgical system of claim 2, wherein the conduit is operable to transmit signals between the tool assembly and the console.

4. The surgical system of claim 2, wherein the conduit is operable to provide irrigation to the tool assembly from the console.

5. The surgical system of claim 2, wherein the conduit is operable to transmit power from the console to the tool assembly.

6. The surgical system of claim 1, wherein the tool assembly includes a motor that is operable to drive one or more attachments coupled with the tool assembly during operation.

7. The surgical system of claim 1, wherein the tool assembly includes a battery to power the tool assembly.

8. The surgical system of claim 1, wherein the tool assembly is wirelessly connected to the console.

9. The surgical system of claim 1, wherein the tool assembly is configured to be packed individually in single use, sterile packaging.

10. The surgical system of claim 1, wherein the one or more actions performed by the tool assembly includes drilling, reaming, and/or sculpting for bones, cartilage, screws, and/or implants.

11. The surgical system of claim 1, wherein the one or more actions performed by the tool assembly includes pin and wire placement for fixation.

12. The surgical system of claim 1, wherein the one or more actions performed by the tool assembly includes sawing for osteotomy of small bones.

13. The surgical system of claim 1, wherein the tool assembly includes one or more attachments, wherein the tool assembly is operable to detachably couple with each of the one or more attachments to perform different actions during operation.

14. The surgical system of claim 13, wherein the one or more attachments includes a drill bit, a drill, a saw, a burr, a minimally invasive procedure tool, and/or a saw blade.

15. The surgical system of claim 13, wherein each of the one or more attachments is disposable and sterile.

16. The surgical system of claim 1, wherein the tool assembly is disposable and sterile.

17. The surgical system of claim 1, further comprising a control component operable to be coupled with the console, the control component operable to receive input from an operator to cause the tool assembly to perform one or more actions.

18. The surgical system of claim 17, wherein the control component is wirelessly connected to the console.

19. The surgical system of claim 17, wherein the control component is coupled with the console via a conduit.

20. The surgical system of claim 17, wherein the control component is operable to convert mechanical force exerted by an operator's foot into an electrical signal that controls the tool assembly during operation.
